(19) United States
(12) Patent Application Publication
(10) Pub. No.: US 2026/0055115 A1
(43) Pub. Date: Feb. 26, 2026

(54) NOVEL ANTHRAQUINONE-BASED NIR ABSORBERS

(71) Applicants: BASF SE, Ludwigshafen am Rhein (DE); Max-Planck-Gesellschaft zur Förderung der Wissenschaften e.V., München (DE)

(72) Inventors: Klaus MUELLEN, Mainz (DE); Zehua WU, Mainz (DE); Hans REICHERT, Basel (CH); Hans Helmut REICHELT, Neustadt (DE)

(21) Appl. No.: 19/104,776

(22) PCT Filed: Aug. 16, 2023
(86) PCT No.: PCT/EP2023/072550

§ 371 Date: Feb. 19, 2025

(30) Foreign Application Priority Data
Aug. 22, 2022 (EP) 22191548.1

(51) Int. Cl. C07D 487/04; C09B 5/62; C09D 11/03

(52) U.S. Cl. CPC C07D 487/04; C09B 5/62; C09D 11/03

(57) ABSTRACT

The present invention relates to compounds of the formula (I′), especially compounds of formula (I), a process for their preparation and their use as almost colourless IR absorbers, for optical filter applications, especially for plasma display panels, or for laser welding of plastics. The compounds may be used in compositions for inks, paints and plastics, especially in a wide variety of printing systems and are particularly well-suited for security applications, or for brand protection; or as marker for liquid. Compounds of formula (I′), especially compounds of formula (I), represent almost colourless IR absorbers, which exhibit high resistance against chemicals and solvents as well as good light stability and good thermal stability. Due to their unique application properties they can be advantageously employed as IR absorbers for laser writing, heat shielding, security printing, the laser-welding of plastics and as marker for liquids.

The present invention relates to compounds of formula (I′), especially compounds of formula (I), a process for their preparation and their use as almost colourless IR absorbers, for optical filter applications, especially for plasma display panels, or for laser welding of plastics. The compounds may be used in compositions for inks, paints and plastics, especially in a wide variety of printing systems and are particularly well-suited for security applications, or for brand protection; or as marker for liquid. Compounds of formula (I′), especially compounds of formula (I), represent almost colourless IR absorbers, which exhibit high resistance against chemicals and solvents as well as good light stability and good thermal stability. Due to their unique application properties they can be advantageously employed as IR absorbers for laser writing, heat shielding, security printing, the laser-welding of plastics and as marker for liquids.

### TECHNICAL BACKGROUND

EPO460496B1 relates to anthraquinone dyes of the formula

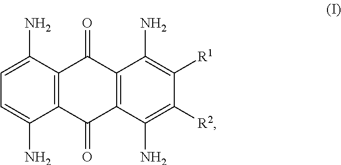

where R¹ and R² are each cyano or together a radical of the formula

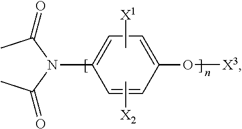

where n is 0 or 1, X¹ and X² are identical or different and each is independently of the other hydrogen, C₁-C₄-alkyl or halogen, and X³ is hydrogen or C₁-C₂₆-alkyl which may be interrupted by one or more oxygen atoms in ether function and substituted by cyano, halogen, hydroxyl, amino, vinyl, C₁-C₆-alkanoyloxy, C₁-C₄-alkenoyloxy or C₃-C₄-haloalkenoyloxy, and their use for the preparation of liquid-crystalline polymers.

EP1672052A1 relates to a method for monitoring degradation of lubricating oils; said method comprising steps of (a) adding to a lubricating oil at least one compound having formula

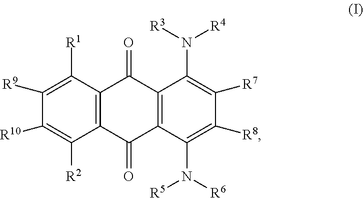

wherein R¹ and R² independently are hydrogen, hydroxy, OR¹¹, amino or NR¹¹R¹²; R³ and R⁵ independently are alkyl, aryl, aralkyl, heteroalkyl or heterocyclic; R⁴ and R⁶ independently are hydrogen or alkyl; R⁷, R⁸, R⁹ and R¹⁰ independently are cyano, nitro, amide, carboxyl, ester, alkyl or hydrogen; R¹¹ is alkyl, aryl, aralkyl, heteroalkyl, heterocyclic or alkanoyl; R¹² is hydrogen or alkyl; provided that said at least one compound of formula (I) has at least one substituent selected from among cyano, nitro, hydroxy, hydroxyalkyl, amide, carboxyl, ester and unsaturated alkyl; and
(b) measuring a spectroscopic property of the oil to determine degradation of said at least one compound.

The need for almost colorless NIR absorbers with sufficient stability in the respective applications is high. It has now been found, surprisingly, that compounds of formula (I′), especially compounds of formula (I) represent almost colourless IR absorbers, which exhibit high resistance against chemicals and solvents as well as good light stability and good thermal stability. Due to their unique application properties they can be advantageously employed as IR absorbers for laser writing, heat shielding, security printing, the laser-welding of plastics and as marker for liquids, especially oils.

### SUMMARY OF THE INVENTION

Thus, in a first aspect, the invention relates to compounds of the formula

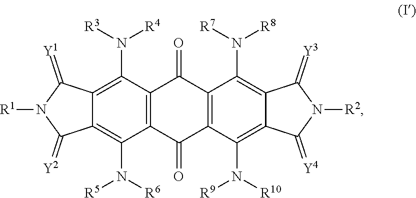

wherein
Y¹, Y², Y³ and Y⁴ are O, or one of Y¹ and Y² is O and the other is NR¹³ and one of Y³ and Y⁴ is O and the other is NR¹³, wherein R¹³ has the meaning of R¹; wherein R¹, R², R³, R⁴, R⁵, R⁸, R⁷, R⁸, R⁹ and R¹⁰ are defined below.

Examples of compounds of formula (I′), wherein one of Y¹ and Y² is O and the other is NR¹³ and one of Y³ and Y⁴ is O and the other is NR¹³ are compounds of formula

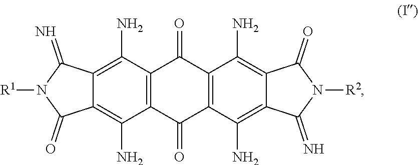

wherein R¹ and R² are identical and are selected from linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹¹, wherein R¹¹ is C₁-C₁₂-alkyl. The compounds of formula (I″) can be prepared in analogy to known methods. Reference is made, for example, to U.S. Pat. No. 4,491,666 and JP58219262.

Compounds of formula (I′), wherein Y¹, Y², Y³ and Y⁴ are O, i.e. compounds of the formula

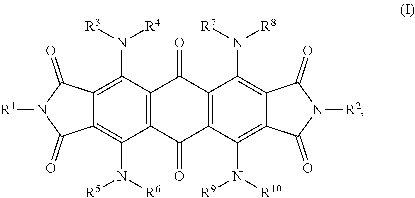

are preferred, wherein
R¹ and R² are independently of each other selected from hydrogen, C₁-C₂₄-alkyl, C₁-C₂₄-haloalkyl, C₃-C₂₄-cycloalkyl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with one or more substituents R¹¹, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from O, S and NR¹²;
R³, R⁴, R⁵, R⁶, R⁷, R⁸, R⁹ and R¹⁰ are independently of each other selected from hydrogen, C₁-C₂₄-alkyl, C₁-C₂₄-haloalkyl, C₃-C₂₄-cycloalkyl, heterocycloalkyl, hetaryl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with one or more substituents R^{11′}, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from O, S and NR^{12′};
each R¹¹ is selected from C₁-C₂₄-alkyl, C₁-C₂₄-fluoroalkyl, C₁-C₂₄-alkoxy, fluorine, chlorine or bromine;
each R^{11′} is selected from C₁-C₂₄-alkyl, C₁-C₂₄-fluoroalkyl, C₁-C₂₄-alkoxy, fluorine, chlorine or bromine; and
R¹² and R^{12′} are independently of each other hydrogen, C₁-C₂₀-alkyl, C₃-C₂₄-cycloalkyl, heterocycloalkyl, hetaryl or C₆-C₁₀-aryl.

In a further aspect, the invention provides a printing ink formulation for security printing, comprising at least one compound of the formula (I′), especially at least one compound of the formula (I), as defined above and below.

In a further aspect, the invention provides a security document, comprising a substrate and at least one compound of the formula (I′), especially at least one compound of the formula (I), as defined above and below.

In a further aspect, the invention provides a security document, obtainable by a printing process, wherein a printing ink formulation is employed that comprises at least one compound of the general formula (I′), at least one compound of the formula (I), as defined above and below.

In one embodiment R¹ and R² are hydrogen.

In another embodiment R¹ and R² are preferably selected from a linear C₁-C₂₄-alkyl group, a branched C₃-C₂₄-alkyl group, a C₅-C₈-cycloalkyl group, a phenyl group, or a phenyl-C₁-C₁₀-alkylene group, where the rings of cycloalkyl, phenyl and phenyl-alkylene in the three last-mentioned radicals are unsubstituted or substituted by 1, 2 or 3 identical or different substituents R¹¹, wherein R¹¹ is defined above and is preferably C₁-C₁₂-alkyl, or C₁-C₁₂-alkoxy. More preferably R¹ and R² are selected from a linear C₁-C₂₄-alkyl group, a branched C₃-C₂₄-alkyl group, a C₅-C₈-cycloalkyl group, or a phenyl group, wherein the two last-mentioned substituents are substituted by one, two or three C₁-C₆-alkyl substituents, or C₁-C₆-alkoxy substituents.

Even more preferably, R¹ and R² are selected from the group consisting of C₅-C₈-cycloalkyl, such as, for example, cyclopentyl, cyclohexyl, methylcyclohexyl, dimethylcyclohexyl, cycloheptyl and cyclooctyl, that is unsubstituted or substituted by one, two or three C₁-C₆-alkyl substituents; linear C₄-C₂₀-alkyl; a radical of formula (A.1); a radical of formula (A.2); a radical of formula (A.3); a radical of formula (B.1); or a radical of formula (B.2)

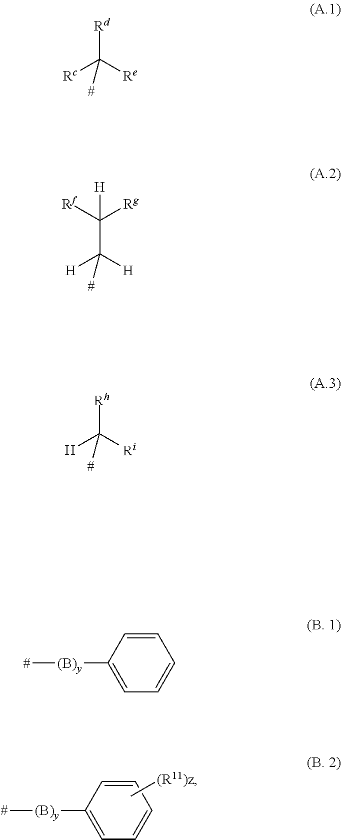

in which
# represents the bonding site to the imide nitrogen atom; R^{c}, R^{d} and R^{e}, in formula (A.1) are independently selected from C₁-C₁₇-alkyl, where the sum of the carbon atoms of the R^{c}, R^{d} and R^{e} radicals is an integer from 3 to 23;
R^{f} and R^{g}, in formula (A.2) are independently selected from C₁-C₁₇-alkyl, where the sum of the carbon atoms of the R^{f} and R^{g} radicals is an integer from 2 to 22;
R^{h} and Rⁱ, in formula (A.3), independently from each other are selected from C₁-C₁-alkyl, where the sum of the carbon atoms of the R^{h} and Rⁱ radicals is an integer from 3 to 23; B, where present in formulae (B.1) and (B.2), is a C₁-C₁₀-alkylene group;
y is 0, 1 or 2;
R¹¹ is independently of one another selected from C₁-C₂₄-alkyl, C₁-C₂₄-fluoroalkyl, C₁-C₂₄-alkoxy, fluorine, chlorine or bromine;
z in formula (B.2) is 1, 2 or 3.

Among the radicals of formulae (A.1), (A.2) and (A.3), the radical of formula (A.3) is preferred. In the context of the radical (A.3), R^{h} and Rⁱ, independently of each other, are preferably selected from linear C₂-C₁₀-alkyl.

Among the radicals of formulae (B.1) and (B.2), in one embodiment those are preferred, in 10 which y is 0, i.e. the variable B is absent. Irrespectively of its occurrence, R¹¹ is preferably selected from C₁-C₂₄-alkyl, more preferably linear C₁-C₁₀-alkyl or branched C₃-C₁₀-alkyl, especially isopropyl or tert-butyl. In particular, the radical of formula (B.2) is preferred. Most preferably, R¹ and R² are a radical of formula (B.2), wherein (B)_{y} is absent. Specific examples of radicals of formula (B.2) are 2,6-dimethylphenyl, 2,4-di(tert-butyl)phenyl, 2,6-diisopropylphenyl or 2,6-di(tert-butyl)phenyl.

Among the radicals of formulae (B.1) and (B.2), in another embodiment those are preferred, in which y is 1, or 2. Irrespectively of its occurrence, R¹¹ is preferably selected from C₁-C₁₂-alkyl, more preferably C₁-C₆-alkyl, or C₁-C₁₂-alkoxy, more preferably linear C₁-C₆-alkoxy. In particular, the radicals of formula (B.1) and (B.2) are preferred. Specific examples of radicals of formula (B.1) and (B.2) are benzyl, or 4-methoxy-2-phenethyl.

In one embodiment R³, R⁴, R⁵, R⁶, R⁷, R⁸, R⁹ and R¹⁰ are preferably selected from a linear C₁-C₂₄-alkyl group, a branched C₃-C₂₄-alkyl group, a C₅-C₈-cycloalkyl group, a phenyl group, or a phenyl-C₁-C₁₀-alkylene group, where the rings of cycloalkyl, phenyl and phenyl-alkylene in the three last-mentioned radicals are unsubstituted or substituted by 1, 2 or 3 identical or different substituents R^{11′}, wherein R^{11′} is defined above and is preferably C₁-C₁₂-alkyl, or C₁-C₁₂-alkoxy. More preferably R¹ and R² are selected from a C₁-C₂₄-alkyl group, a C₅-C₈-cycloalkyl group, or a phenyl group, wherein the two last-mentioned substituents are substituted by one, two or three C₁-C₆-alkyl substituents.

Even more preferably, R³, R⁴, R⁵, R⁸, R⁷, R⁸, R⁹ and R¹⁰ are selected from the group consisting of C₅-C₈-cycloalkyl, such as, for example, cyclopentyl, cyclohexyl, methylcyclohexyl, dimethylcyclohexyl, cycloheptyl and cyclooctyl, that is unsubstituted or substituted by one, two or three C₁-C₆-alkyl substituents; C₁-C₁₂-alkyl;

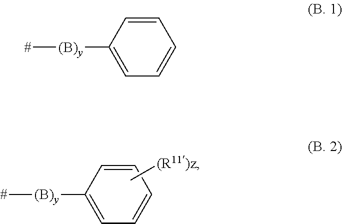

in which
B, where present in formulae (B.1) and (B.2), is a C₁-C₁₀-alkylene group;
y is 0, 1 or 2;
R^{11′} is independently of one another selected from C₁-C₂₄-alkyl, C₁-C₂₄-fluoroalkyl, C₁-C₂₄-alkoxy, fluorine, chlorine or bromine;
z in formula (B.2) is 1, 2 or 3.

Among the radicals of formulae (B.1) and (B.2), those are preferred, in which y is 0, i.e. the variable B is absent. Irrespectively of its occurrence, R¹¹ is preferably selected from C₁-C₂₄-alkyl, more preferably linear C₁-C₁₀-alkyl or branched C₃-C₁₀-alkyl, especially methyl, isopropyl, n-butyl, or tert-butyl. In particular, the radical of formula (B.2) is preferred. Most preferably, R³, R⁴, R⁵, R⁶, R⁷, R⁸, R⁹ and R¹⁰ are a radical of formula (B.2), wherein (B)_{y} is absent. Specific examples of radicals of formula (B.2) are 2,4,6-trimethylphenyl, 2,6-diisopropylphenyl, or 4-n-butylphenyl.

In another preferred embodiment R³, R⁵, R⁷ and R⁹ are hydrogen and R⁴, R⁶, R⁸ and R¹⁰ are selected from a linear C₁-C₂₄-alkyl group, a branched C₃-C₂₄-alkyl group, a C₅-C₈-cycloalkyl group, a phenyl group, or a phenyl-C₁-C₁₀-alkylene group, where the rings of cycloalkyl, phenyl and phenyl-alkylene in the three last-mentioned radicals are unsubstituted or 15 substituted by 1, 2 or 3 identical or different substituents R^{11′}, wherein R^{11′} is defined above and is preferably C₁-C₁₂-alkyl, or C₁-C₁₂-alkoxy. More preferably R¹ and R² are selected from a C₄-C₂₀-alkyl group, a C₅-C₈-cycloalkyl group, or a phenyl group, wherein the two last-mentioned substituents are substituted by one, two or three C₁-C₆-alkyl substituents.

Even more preferably, R⁴, R⁶, R⁸ and R¹⁰ are selected from the group consisting of C₅-C₈-cycloalkyl, such as, for example, cyclopentyl, cyclohexyl, methylcyclohexyl, dimethylcyclohexyl, cycloheptyl and cyclooctyl, that is unsubstituted or substituted by one, two or three C₁-C₆-alkyl substituents; C₁-C₁₂-alkyl;

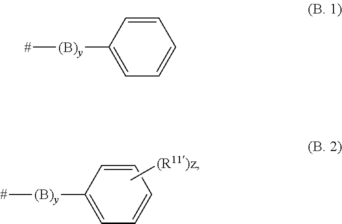

in which
B, where present in formulae (B.1) and (B.2), is a C₁-C₁₀-alkylene group;
y is 0, 1 or 2;
R^{11′} is independently of one another selected from C₁-C₂₄-alkyl, C₁-C₂₄-fluoroalkyl, C₁-C₂₄-alkoxy, fluorine, chlorine or bromine;
z in formula (B.2) is 1, 2 or 3.

Among the radicals of formulae (B.1) and (B.2), those are preferred, in which y is 0, i.e. the variable B is absent. Irrespectively of its occurrence, R^{11′} is preferably selected from C₁-C₂₄-alkyl, more preferably linear C₁-C₁₀-alkyl or branched C₃-C₁₀-alkyl, especially methyl, isopropyl, n-butyl, or tert-butyl. In particular, the radical of formula (B.2) is preferred. Most preferably, R⁴, R⁶, R⁸ and R¹⁰ are a radical of formula (B.2), wherein (B)_{y} is absent. Specific examples of radicals of formula (B.2) are 2,4,6-trimethylphenyl, 2,6-diisopropylphenyl, or 4-n-butylphenyl.

The compounds of the general formula (I) have at least one of the following advantageous properties:
good fastness to chemicals, in particular fastness to bleaching with hypochlorite and fastness to solvents (like toluene, acetone or dichloromethane),
good fastness to boiling water,
good fastness to light,
colourlessness (i.e. minimal absorption in the VIS range of the spectrum (from 400 to 700 nm))
good heat stability,
high compatibility with a multiplicity of formulations, in particular printing ink formulations used in security printing and thermoplastic polymer formulations used for laser-welding.

For definition and description of fastness requirements in banknote printing see e.g. “Chemical and Physical Resistance” in “Extract of the ANNEX 13 of the Technical Specification for Euro banknote production” (European Central Bank; July 2004).

The compounds of general formula (I) can be used inter alia for security printing, for brand protection, as marker for liquids, especially oils, invisible and/or IR readable bar codes, the laser-welding of plastics, the curing of surface-coatings using IR radiators, the drying and curing of print, the fixing of toners on paper or plastics, optical filters for plasma display panels, laser marking of paper or plastics, the heating of plastics preforms, and for heat shielding applications.

The compounds of formula (I), especially the compounds of formula (I), wherein R¹ and R² and/or R⁴, R⁶, R⁸ and R¹⁰ are different from hydrogen, may provide several benefits, in particular high solubility and/or (photo)stability in the application medium. Moreover, certain compounds of formula (I) are outstandingly suitable as IR absorber absorbing light comprising a wavelength of 680 to 1000 nm due to their good solubility in the application medium and the high absorption. In addition, the compounds of formula (I), especially the compounds of formula (I), wherein R¹ and R² and/or R⁴, R⁶, R⁸ and R¹⁰ are different from hydrogen, may be outstandingly suitable as marker for liquids, especially oils, such as mineral oils due to its favorable application properties such as good solubility in the liquids, high absorption, good photo, storage stability and good detectability even in very small amounts in the correspondingly marked liquids.

### DETAILED DESCRIPTION OF THE INVENTION

Here and throughout the specification, the term “near-infrared light” denotes light that ranges from 680 to 1000 nm.

Here and throughout the specification, the term “visible light” denotes light that ranges from approximately 380 nm to 740 nm.

The term “C₁-Cₙ-alkyl” denotes a group of linear or branched saturated hydrocarbon radicals having from 1 to n carbon atoms. For example, the term C₁-C₂₄-alkyl denominates a group of linear or branched saturated hydrocarbon radicals having from 1 to 24 carbon atoms, while the term C₁-C₄-alkyl denominates a group of linear or branched saturated hydrocarbon radicals having from 1 to 4 carbon atoms, the term C₅-C₂₀ alkyl denominates a group of linear or branched saturated hydrocarbon radicals having from 5 to 20 carbon atoms and the term C₆-C₂₀-alkyl denominates a group of linear or branched saturated hydrocarbon radicals having from 6 to 20 carbon atoms. Examples of alkyl include but are not limited to methyl, ethyl, n-propyl, isopropyl, n-butyl, sec-butyl, isobutyl, tert-butyl, 2-methylpropyl (isopropyl), 1,1-dimethylethyl (tert-butyl), pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, 1,1,3,3-tetramethylbutyl (tert-octyl), nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl and in case of nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl their isomers, in particular mixtures of isomers such as “isononyl”, “isodecyl”. Examples of C₁-C₄-alkyl are for example methyl, ethyl, propyl, 1-methylethyl, butyl, 1-methylpropyl, 2-methylpropyl or 1,1-dimethylethyl.

The term “C₁-C₂₄-haloalkyl” as used herein denotes straight-chain or branched C₁-C₂₄ alkyl as defined above, where some or all of the hydrogen atoms in these groups may be replaced by halogen atoms as mentioned above. Examples for C₁-C₂-haloalkyl are chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl.

The term “C₁-C₂₄-fluoroalkyl” as used herein denotes straight-chain or branched C₁-C₂₄ alkyl as defined above, where some or all of the hydrogen atoms in these groups may be replaced by fluorine above. Examples for C₁-C₂-fluoroalkyl are fluoromethyl, difluoromethyl, trifluoromethyl, 1-fluoroethyl, 2-fluoroethyl, 2,2-difluoroethyl, 2,2,2-trifluoroethyl and pentafluoroethyl. The term “C₁-C₂₄-alkoxy” as used herein denotes straight-chain or branched C₁-C₂₄ alkyl as defined above bound to the remainder of the molecule through an oxygen. Examples for C₁-C₄-alkoxy are methoxy, ethoxy, n-propoxy, 1-methylethoxy, butoxy, 1-methylpropoxy, 2-methylpropoxy and 1,1-dimethylethoxy.

The term “C₃-C₂₄-cycloalky” as used herein denotes a mono-, bi- or tricyclic cycloalkyl radical which is unsubstituted or substituted by one or more radicals R⁷, for example 1, 2, 3 or 4 R⁷ radicals. Examples of C₃-C₂₄-cycloalkyl include but are not limited to cyclopropyl, cyclobutyl, cyclopentyl, cyclohexyl, methylcyclohexyl, dimethylcyclohexyl, cycloheptyl, cyclooctyl, cyclododecyl, cyclohexadecyl and norbornyl (=bicyclo[2.2.1]heptyl).

The term “C₆-C₁₀-aryl” as used herein denotes phenyl or naphthyl.

The term “C₆-C₁₀-aryloxy” as used herein denotes phenoxy and naphthyloxy.

The term “alkylene” or “alkanediyl” as used herein denotes a straight-chain or branched alkyl radical as defined above, wherein one hydrogen atom at any position of the carbon backbone is replaced by one further binding site, thus forming a bivalent moiety.

The term “C₆-C₁₀-aryl-C₁-C₁₀-alkylene” (which may also be referred to as aralkyl) as used herein refers to C₆-C₁₀-aryl-substituted alkyl radicals having at least one unsubstituted or substituted aryl group, as defined herein. The alkyl group of the aralkyl radical may be interrupted by one or more nonadjacent groups selected from O, S and NR¹², wherein R¹² is as defined above. C₆-C₁₀-aryl-C₁-C₁₀-alkylene is preferably phenyl-C₁-C₁₀-alkylene, more preferably phenyl-C₁-C₄-alkylene, for example benzyl, 1-phenethyl, 2-phenethyl, 1-phenprop-1-yl, 2-phenprop-1-yl, 3-phenprop-1-yl, 1-phenbut-1-yl, 2-phenbut-1-yl, 3-phenbut-1-yl, 4-phenbut-1-yl, 1-phenbut-2-yl, 2-phenbut-2-yl, 3-phenbut-2-yl or 4-phenbut-2-yl; preferably benzyl and 2-phenethyl.

The term “heterocycloalkyl” as used herein refers to a mono- or bicyclic saturated or partially unsaturated ring system having 3, 4, 5, 6, 7 or 8 ring members (C₃-C₅-heterocyclyl), comprising besides carbon atoms as ring members, one, two, three or four heteroatoms or heteroatom-containing groups selected from O, N, S, SO and S(O)₂ as ring members.

The term “hetaryl” or “heteroaryl” as used herein refers to heteroaromatic, monocyclic, bicyclic or tricyclic condensed system with 5, 6, 7, 8, 9, 10, 11, 12, 13 or 14 ring members in which at least one of the rings is aromatic and which contains 1, 2, 3 or 4 heteroatoms selected from N, S or O. Monocyclic hetaryl groups are preferably 5- or 6-membered hetaryl groups comprising 1, 2 or 3 heteroatoms selected from O, S or N such as 2-furyl (furan-2-yl), 3-furyl (furan-3-yl), 2-thienyl (thiophen-2-yl), 3-thienyl (thiophen-3-yl), 1H-pyrrol-2-yl, 1H-pyrrol-3-yl, pyrrol-1-yl, imidazol-2-yl, imidazol-1-yl, imidazol-4-yl, pyrazol-1-yl, pyrazol-3-yl, pyrazol-4-yl, pyrazol-5-yl, 3-isoxazolyl, 4-isoxazolyl, 5-isoxazolyl, 3-isothiazolyl, 4-isothiazolyl, 5-isothiazolyl, 2-oxazolyl, 4-oxazolyl, 5-oxazolyl, 2-thiazolyl, 4-thiazolyl, 5-thiazolyl, 1,2,4-oxadiazol-3-yl, 1,2,4-oxadiazol-5-yl, 1,3,4-oxadiazol-2-yl, 1,2,4-thiadiazol-3-yl, 1,2,4-thiadiazol-5-yl, 1,3,4-thiadiazol-2-yl, 4H-[1,2,4]-triazol-3-yl, 1,3,4-triazol-2-yl, 1,2,3-triazol-1-yl, 1,2,4-triazol-1-yl, pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Bicyclic throughout aromatic heteroaryl is 9- or 10-membered and contains 1, 2, 3 or 4 heteroatoms selected from O, S or N. Examples are quinolinyl, isoquinolinyl, indolyl, isoindolyl, indolizinyl, benzofuranyl, isobenzofuranyl, benzothiophenyl, benzoxazolyl, benzisoxazolyl, benzthiazolyl, benzoxadiazolyl, benzothiadiazolyl, benzoxazinyl, benzopyrazolyl, benzimidazolyl, benzotriazolyl, benzotriazinyl.

In a preferred embodiment the present invention is directed to compounds of formula

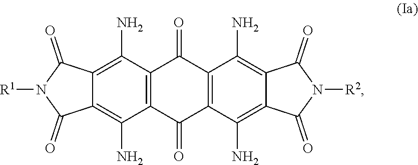

wherein
R¹ and R² are defined above. In said embodiment the present invention is directed to a compound of formula

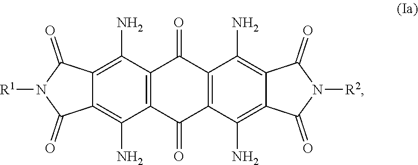

wherein
R¹ and R² are hydrogen, which is pigmentary, and compounds of formula

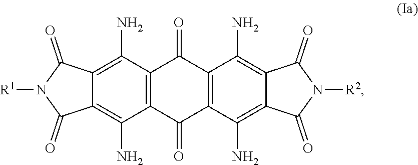

wherein R¹ and R² are different from hydrogen, and which may be soluble in the application medium.

If R¹ and R² are different from hydrogen, they are independently of each other selected from linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹, wherein R¹¹ is defined above, or below and is preferably C₁-C₁₂-alkyl.

More preferably, R¹ and R² are identical and are selected from linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹¹, wherein R¹¹ is C₁-C₁₂-alkyl.

Most preferred, R¹ and R² are preferably identical and are preferably selected from linear, or branched C₃-C₂₄-alkyl, radicals of formula

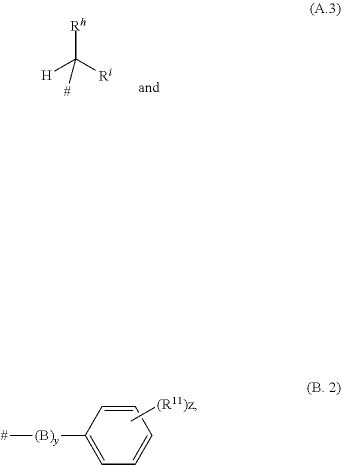

in which
# represents the bonding site to the imide nitrogen atom;
R^{h} and Rⁱ, in formula (A.3), independently from each other are selected from C₁-C_{1B}-alkyl,
where the sum of the carbon atoms of the R^{h} and Rⁱ radicals is an integer from 3 to 25;
B, where present in formulae (B.1) and (B.2), is a C₁-C₁₀-alkylene group;
y is 0 or 1;
R¹¹ is independently of one another selected from C₁-C₂₄-alkyl, C₁-C₂₄-fluoroalkyl, C₁-C₂₄-alkoxy, fluorine, chlorine or bromine;
z in formula (B.2) is 1, 2 or 3.
R^{h} and Rⁱ, independently of each other, are preferably selected from linear C₂-C₁₀-alkyl.

Among the radicals of formulae (B.1) and (B.2), those are preferred, in which y is 0, i.e. the variable B is absent. Irrespectively of its occurrence, R¹¹ is preferably selected from C₁-C₂₄-alkyl, more preferably linear C₁-C₁₀-alkyl or branched C₃-C₁₀-alkyl, especially isopropyl or tert-butyl. In particular, the radical of formula (B.2) is preferred. Most preferably, R¹ and R² are identical and a radical of formula (B.2), wherein (B)_{y} is absent. Specific examples of radicals of formula (B.2) are 2,6-dimethylphenyl, 2,4-di(tert-butyl)phenyl, 2,6-diisopropylphenyl or 2,6-di(tert-butyl)phenyl.

Examples of particularly preferred compounds of formula (Ia) are compounds (3), (4) and (4a) to (4h) described in claim 7.

In another preferred embodiment the present invention is directed to compounds of formula

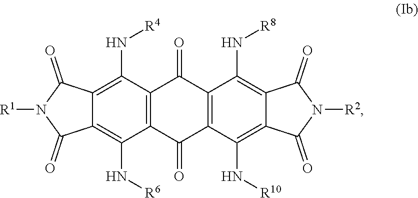

wherein
R⁴, R⁶, R⁸, R¹⁰, R¹ and R² are defined above, or below.

In said embodiment the present invention is directed to compounds of formula

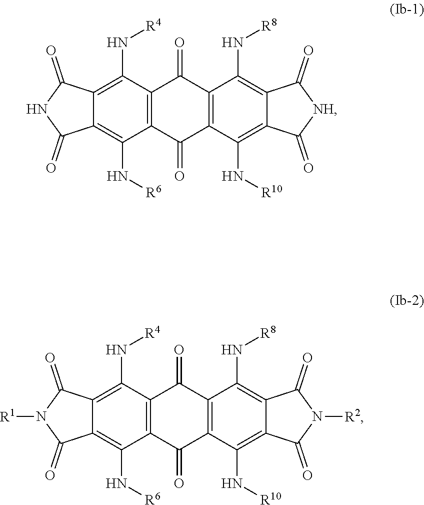

wherein R¹ and R² are different from hydrogen.

If R¹ and R² are different from hydrogen, they are independently of each other selected from linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹, wherein R¹¹ is defined in claim 1 and is preferably C₁-C₁₂-alkyl.

More preferably, R¹ and R² are identical and are selected from hydrogen, linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹, wherein R¹¹ is C₁-C₁₂-alkyl.

Most preferred, R¹ and R² are preferably identical and are preferably selected from linear C₁-C₂₄-alkyl, radicals of formula

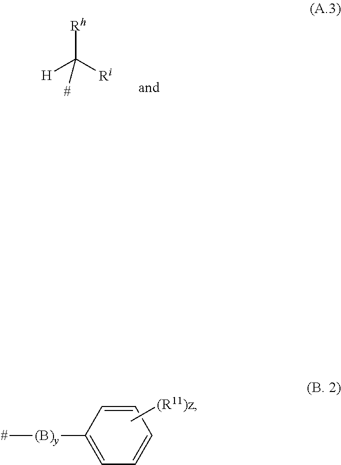

in which
# represents the bonding site to the imide nitrogen atom;
R^{h} and Rⁱ, in formula (A.3), independently from each other are selected from C₁-C₁₈-alkyl, where the sum of the carbon atoms of the R^{h} and Rⁱ radicals is an integer from 3 to 25;
B, where present in formulae (B.1) and (B.2), is a C₁-C₁₀-alkylene group;
y is 0 or 1;
R¹¹ is independently of one another selected from C₁-C₂₄-alkyl, C₁-C₂₄-fluoroalkyl, C₁-C₂₄-alkoxy, fluorine, chlorine or bromine;
z in formula (B.2) is 1, 2 or 3.

R^{h} and Rⁱ, independently of each other, are preferably selected from linear C₂-C₁₀-alkyl.

Among the radicals of formulae (B.1) and (B.2), those are preferred, in which y is 0, i.e. the variable B is absent. Irrespectively of its occurrence, R¹¹ is preferably selected from C₁-C₂₄-alkyl, more preferably linear C₁-C₁₀-alkyl or branched C₃-C₁₀-alkyl, especially isopropyl or tert-butyl. In particular, the radical of formula (B.2) is preferred. Most preferably, R¹ and R² are identical and a radical of formula (B.2), wherein (B)_{y} is absent. Specific examples of radicals of formula (B.2) are 2,6-dimethylphenyl, 2,4-di(tert-butyl)phenyl, 2,6-diisopropylphenyl or 2,6-di(tert-butyl)phenyl.

Preferably, R⁴, R⁶, R⁸ and R¹⁰ are independently of each other selected from hydrogen, C₁-C₂₄-alkyl, C₃-C₂₄-cycloalkyl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with 1, 2 or 3 identical or different substituents R^{11′}, wherein R^{11′} is defined in claim 1 and is preferably C₁-C₁₂-alkyl.

More preferably, R⁴, R⁶, R⁸ and R¹⁰ are identical and are selected from C₁-C₂₄-alkyl, C₃-C₂₄-cycloalkyl, phenyl and phenyl-C₁-C₄-alkylene, where the rings of cycloalkyl, phenyl, and phenyl-alkylene are unsubstituted or substituted with 1, 2 or 3 identical or different substituents R^{11′}, wherein R^{11′} is C₁-C₁₂-alkyl.

Most preferred, R⁴, R⁶, R⁸ and R¹⁰ are preferably identical and are preferably selected from linear C₁-C₂₄-alkyl, radicals of formula

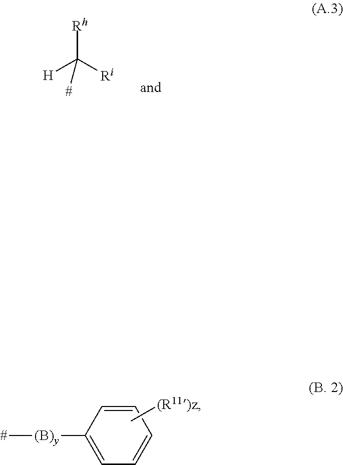

in which
# represents the bonding site to the imide nitrogen atom; R^{h} and Rⁱ, in formula (A.3), independently from each other are selected from C₁-C₁₈-alkyl,
where the sum of the carbon atoms of the R^{h} and Rⁱ radicals is an integer from 3 to 25;
B, where present in formulae (B.1) and (B.2), is a C₁-C₁₀-alkylene group;
y is 0 or 1;
R^{11′} is independently of one another selected from C₁-C₂₄-alkyl, C₁-C₂₄-fluoroalkyl, C₁-C₂₄-alkoxy, fluorine, chlorine or bromine;
z in formula (B.2) is 1, 2 or 3.

R^{h} and Rⁱ, independently of each other, are preferably selected from linear C₂-C₁₀-alkyl.

Among the radicals of formulae (B.2), those are preferred, in which y is 0, i.e. the variable B is absent. Irrespectively of its occurrence, R^{11′} is preferably selected from C₁-C₂₄-alkyl, more preferably linear C₁-C₁₀-alkyl or branched C₃-C₁₀-alkyl, especially isopropyl or tert-butyl. In particular, the radical of formula (B.2) is preferred. Most preferably, R⁴, R⁶, R⁸ and R¹⁰ are identical and a radical of formula (B.2), wherein (B)_{y} is absent. Specific examples of radicals of formula (B.2) are 2,6-dimethylphenyl, 2,4-di(tert-butyl)phenyl, 2,6-diisopropylphenyl or 2,6-di(tert-butyl)phenyl.

Examples of particularly preferred compounds of formula (Ib) are compounds (5a1) to (5g10) described in claim 7.

The compounds of the present invention can be prepared by using routine techniques familiar to a skilled person. In particular, the compounds of the formula (I) can be prepared according to the following routes or as described in the experimental part of this application.

A further aspect of the present invention is a process for the preparation of a compound of formula

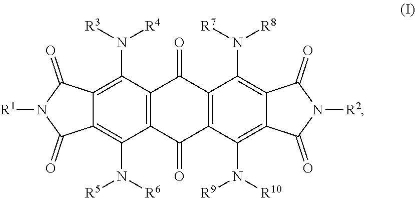

wherein R¹, R², R³, R⁴, R⁵, R⁶, R⁷, R⁸, R⁹ and R¹⁰ are defined above, comprising the following steps:
i) heating a compound of formula

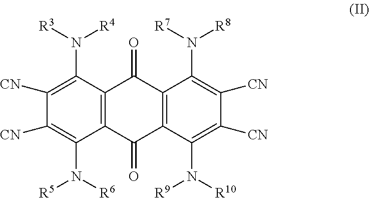

in H₂SO₄, preferably at a temperature of from 50 to 200° C. for 0.5 to 48 h, to obtain a compound of formula (I), wherein R¹ and R² are hydrogen, and
ii) optionally reacting the compound obtained in step i) with a compound R¹—X¹ in the presence of a base to obtain a compound of formula (I), wherein X¹ is Cl, Br, or I, and R¹ and R² are different from hydrogen.

Alternatively, the compound (I) obtained in step i), wherein R¹ and R² are hydrogen, can be converted into the dianhydride of the formula

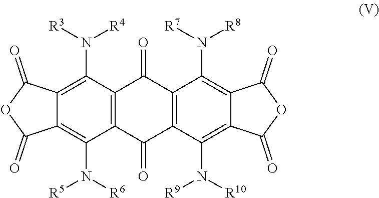

by conventional means and subsequently reacted with a compound R¹—NH₂ to obtain a compound of formula (I), wherein R¹ and R² are different from hydrogen.

The compound of formula

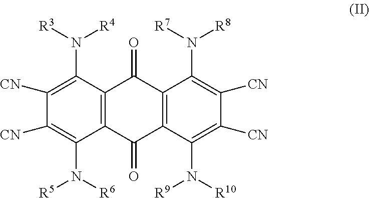

is obtained by reacting a compound of formula

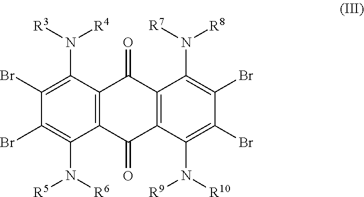

with CuCN in a solvent at elevated temperature, wherein R³, R⁴, R⁵, R⁶, R⁷, R⁸, R⁹ and R¹⁰ are defined above, or below.

The compound of formula

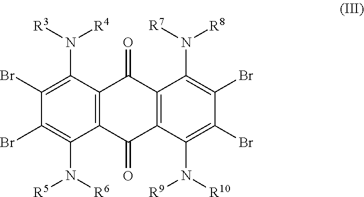

is obtained by reacting a compound of formula

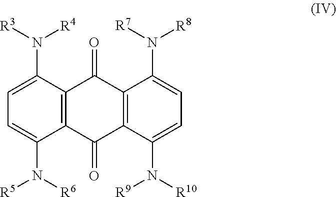

with Br₂ in a solvent, such as, for example, acetic acid, at elevated temperature, such as, for example, at a temperature of 70 to 150° C. for 2 to 12 h, wherein R³, R⁴, R⁵, R⁶, R⁷, R⁸, R⁹ and R¹⁰ are defined above, or below.

All reactions are typically carried out in the absence of oxygen and moisture. As a rule, the reaction mixtures are worked up in the customary manner, for example by mixing with water, separating the phases, and, if appropriate purifying the crude product by chromatography. If the end product is obtained as solids, they may be purified by recrystallization.

The compounds of the formula (I) according to the invention may be incorporated without any problem into organic and inorganic materials and are therefore suitable for a whole series of end uses, some of which will be listed by way of example below.

The IR absorbers of formula (I) can also be used in the form of a mixture, comprising at least one compound of the general formula (I) and at least one further IR absorber different from compounds of the general formula (I). Suitable further IR absorbers are in principle all known classes of IR absorbers that are compatible with the compounds of the general formula (I).

Preferred further IR absorbers are selected from metal dithiolene, polymethines, phthalocyanines, naphthalocyanines, quinone-diimmonium salts, aminium salts, rylenes, inorganic IR absorbers and mixtures thereof. Further polymethine IR absorbers are preferably selected from cyanines, squaraines, croconaines and mixtures thereof. Further inorganic IR absorbers are preferably selected from indium tin oxide, antimony tin oxide, lanthanum hexaboride, tungsten bronzes, copper salts etc.

The afore-mentioned IR absorbers of the general formula (I) and IR absorber mixtures are especially suitable for security printing.

Security printing is the field that deals with the printing of items such as currency, passports, tamper-evident labels, stock certificates, postage stamps, identity cards, etc. The main goal of security printing is to prevent forgery, tampering or counterfeiting.

All security documents are required to have good stability and durability. Furthermore, it is essential that the documents nevertheless should have a reasonable life time, ideally of some years, despite suffering the afore-mentioned conditions. During this time, the documents, and thus the inks on them (including invisible security markings), should be resistant to fading or colour change. Hence, any ink used in a security printing process should, when cured, be robust, water-resistant, resistant to various chemicals and flexible. Moreover, as certain states are moving away from the use of paper as the substrate for bank notes, the employed printing ink formulations should be useable on plastics as well as paper. It has now been found that the compounds of the general formula (I) because of their unique application properties are especially suitable for printing ink formulations that are employed for security printing and in particular for bank notes.

In security printing, the IR absorber is added to a printing ink formulation. Suitable printing inks are water-based, oil-based or solvent-based printing inks, based on pigment or dye, for inkjet printing, flexographic printing, screen printing, intaglio printing, offset printing, laser printing or letterpress printing and for use in electrophotography. Printing inks for these printing processes usually comprise solvents, binders, and also various additives, such as plasticizers, antistatic agents or waxes. Printing inks for offset printing and letterpress printing are usually formulated as high-viscosity paste printing inks, whereas printing inks for flexographic printing and intaglio printing are usually formulated as liquid printing inks with comparatively low viscosity.

In the context of the present invention, the expression “printing ink” also encompasses formulations that in addition to at least one IR absorber of the general formula (I) comprise a colorant. The expression “printing ink” also encompasses printing lacquers that comprise no colorant.

The printing ink formulation for security printing according to the invention preferably comprises
a) at least one compound of the general formula (I) as defined above,
b) a polymeric binder,
c) a solvent,
d) optionally at least one colorant, and
e) optionally at least one further additive.

Suitable components of printing inks are conventional and are well known to those skilled in the art. Examples of such components are described in “Printing Ink Manual”, fourth edition, Leach R. H. et al. (eds.), Van Nostrand Reinhold, Wokingham, (1988). Details of printing inks and their formulation are also disclosed in “Printing Inks”-Ullmann's Encyclopedia of Industrial Chemistry, Sixth Edition, 1999 Electronic Release. A formulation of an IR-absorbing intaglio ink formulation is described in US 20080241492 A1. The disclosure of the afore-mentioned documents is incorporated herein by reference.

The printing ink formulation according to the invention contains in general from 0.0001 to 25% by weight, preferably from 0.001 to 15% by weight, in particular from 0.01 to 5% by weight, based on the total weight of the printing ink formulation, of component a).

The compounds of the general formula (I) are present in the printing ink formulation in dissolved form or in solid form (in a finely divided state).

The printing ink formulation according to the invention contains in general from 5 to 74% by weight, preferably from 10 to 60% by weight, more preferably from 15 to 40% by weight, based on the total weight of the printing ink formulation, of component b).

Suitable polymeric binders b) for the printing ink formulation according to the invention are for example selected from natural resins, phenol resin, phenol-modified resins, alkyd resins, polystyrene homo- and copolymers, terpene resins, silicone resins, polyurethane resins, urea-formaldehyde resins, melamine resins, polyamide resins, polyacrylates, polymethacrylates, chlorinated rubber, vinyl ester resins, acrylic resins, epoxy resins, nitrocellulose, hydrocarbon resins, cellulose acetate, and mixtures thereof.

The printing ink formulation according to the invention can also comprise components that form a polymeric binder by a curing process. Thus, the printing ink formulation according to the invention can also be formulated to be energy-curable, e.g. able to be cured by UV light or EB (electron beam) radiation. In this embodiment, the binder comprises one or more curable monomers and/oligomers. Corresponding formulations are known in the art and can be found in standard textbooks such as the series “Chemistry & Technology of UV & EB Formulation for Coatings, Inks & Paints”, published in 7 volumes in 1997-1998 by John Wiley & Sons in association with SITA Technology Limited.

Suitable monomers and oligomers (also referred to as prepolymers) include epoxy acrylates, acrylated oils, urethane acrylates, polyester acrylates, silicone acrylates, acrylated amines, and acrylic saturated resins. Further details and examples are given in “Chemistry & Technology of UV & EB Formulation for Coatings, Inks & Paints”, Volume II: Prepolymers & Reactive Diluents, edited by G Webster.

If a curable polymeric binder is employed, it may contain reactive diluents, i.e. monomers which act as a solvent and which upon curing are incorporated into the polymeric binder. Reactive monomers are typically chosen from acrylates or methacrylates, and can be monofunctional or multifunctional. Examples of multifunctional monomers include polyester acrylates or methacrylates, polyol acrylates or methacrylates, and polyether acrylates or methacrylates.

In the case of printing ink formulations to be cured by UV radiation, it is usually necessary to include at least one photoinitiator to initiate the curing reaction of the monomers upon exposure to UV radiation. Examples of useful photoinitiators can be found in standard textbooks such as “Chemistry & Technology of UV & EB Formulation for Coatings, Inks & Paints”, Volume Ill, “Photoinitiators for Free Radical Cationic and Anionic Polymerisation”, 2nd edition, by J. V. Crivello & K. Dietliker, edited by G. Bradley and published in 1998 by John Wiley & Sons in association with SITA Technology Limited. It may also be advantageous to include a sensitizer in conjunction with the photoinitiator in order to achieve efficient curing.

The printing ink formulation according to the invention contains in general from 1 to 94.9999% by weight, preferably from 5 to 90% by weight, in particular from 10 to 85% by weight, based on the total weight of the printing ink formulation, of a solvent c).

Suitable solvents are selected from water, organic solvents and mixtures thereof. For the purpose of the invention, reactive monomers which also act as solvents are regarded as part of the afore-mentioned binder component b).

Examples of solvents comprise water; alcohols, e.g. ethanol, 1-propanol, 2-propanol, ethylene glycol, propylene glycol, diethylene glycol and ethoxy propanol; esters, e.g. ethyl acetate, isopropyl acetate, n-propyl acetate and n-butyl acetate; hydrocarbons, e.g. toluene, xylene, mineral oils and vegetable oils, and mixtures thereof.

The printing ink formulation according to the invention may contain an additional colorant d). Preferably, the printing ink formulation contains from 0 to 25% by weight, more preferably from 0.1 to 20% by weight, in particular from 1 to 15% by weight, based on the total weight of the printing ink formulation, of a colorant d).

Suitable colorants d) are selected conventional dyes and in particular conventional pigments. The term “pigment” is used in the context of this invention comprehensively to identify all pigments and fillers, examples being colour pigments, white pigments, and inorganic fillers. These include inorganic white pigments, such as titanium dioxide, preferably in the rutile form, barium sulfate, zinc oxide, zinc sulfide, basic lead carbonate, antimony trioxide, lithopones (zinc sulfide+barium sulfate), or coloured pigments, examples being iron oxides, carbon black, graphite, zinc yellow, zinc green, ultramarine, manganese black, antimony black, manganese violet, Paris blue or Schweinfurt green. Besides the inorganic pigments the printing ink formulation of the invention may also comprise organic colour pigments, examples being sepia, gamboge, Cassel brown, toluidine red, para red, Hansa yellow, indigo, azo dyes, anthraquinonoid and indigoid dyes, and also dioxazine, quinacridone, phthalocyanine, isoindolinone, and metal complex pigments. Also suitable are synthetic white pigments with air inclusions to increase the light scattering, such as the Rhopaque® dispersions. Suitable fillers are, for example, aluminosilicates, such as feldspars, silicates, such as kaolin, talc, mica, magnesite, alkaline earth metal carbonates, such as calcium carbonate, in the form for example of calcite or chalk, magnesium carbonate, dolomite, alkaline earth metal sulfates, such as calcium sulfate, silicon dioxide, etc.

The printing ink formulation according to the invention may contain at least one additive e). Preferably, the printing ink formulation contains from 0 to 25% by weight, more preferably from 0.1 to 20% by weight, in particular from 1 to 15% by weight, based on the total weight of the printing ink formulation, of at least one component e).

Suitable additives (component e)) are selected from plasticizers, waxes, siccatives, antistatic agents, chelators, antioxidants, stabilizers, adhesion promoters, surfactants, flow control agents, defoamers, biocides, thickeners, etc. and combinations thereof. These additives serve in particular for fine adjustment of the application-related properties of the printing ink, examples being adhesion, abrasion resistance, drying rate, or slip.

In particular, the printing ink formulation for security printing according to the invention preferably contains
a) 0.0001 to 25% by weight of at least one compound of the general formula (I),
b) 5 to 74% by weight of at least one polymeric binder,
c) 1 to 94.9999% by weight of at least one a solvent,
d) 0 to 25% by weight of at least one colorant, and
e) 0 to 25% by weight of at least one further additive,
wherein the sum of components a) to e) adds up to 100%.

The printing ink formulations according to the invention are advantageously prepared in a conventional manner, for example by mixing the individual components. As mentioned earlier, the IR absorber (I) is present in the printing ink formulations in a dissolved or finely divided solid form. Additional colorants may be employed in the printing ink formulation of the invention or in a separate ink formulation. When additional colorants are to be employed in a separate formulation, the time of application of the printing ink formulation according to the invention is usually immaterial. The printing ink formulation according to the invention can for example be applied first and then be overprinted with conventional printing inks. But it is also possible to reverse this sequence or, alternatively, to apply the printing ink formulation according to the invention in a mixture with conventional printing inks. In every case the prints are readable with suitable light sources.

Primers can be applied prior to the printing ink formulation according to the invention. By way of example, the primers are applied in order to improve adhesion to the substrate. It is also possible to apply additional printing lacquers, e.g. in the form of a covering to protect the printed image. Additional printing lacquers may also be applied to serve aesthetic purposes, or serve to control application-related properties. By way of example, suitably formulated additional printing lacquers can be used to influence the roughness of the surface of the substrate, the electrical properties, or the water-vapour-condensation properties. Printing lacquers are usually applied in-line by means of a lacquering system on the printing machine employed for printing the printing ink formulation according to the invention.

The printing ink formulations according to the invention are also suitable for use in multilayer materials. Multilayer materials are e.g. composed of two or more plastics foils, such as polyolefin foils, metal foils, or metallised plastics foils, which are bonded to one another, by way of example, via lamination or with the aid of suitable laminating adhesives. These composites may also comprise other functional layers, such as odour-barrier layers or water-vapour barriers.

The afore-mentioned IR absorbers of the general formula (I) and IR absorber mixtures are also especially suitable for laser welding of plastics.

The concentration of the IR absorber of the general formula (I) or an IR absorber mixtures is e.g. from 5 to 500 ppm, preferably from 10 to 200 ppm.

In laser welding, plastics components are welded to one another. The plastics components to be fused may have any shape. For example, at least one of the plastics components may be a film.

The compounds of formula (I) are suitable for welding transparent at least translucent plastics materials. The employed plastics materials may be colourless or coloured. In principle, the plastics components to be fused may be composed of the same polymer or of different polymers. Preferably, the plastics components employed for laser welding are selected from thermoplastic polymers. However, it is also possible that neither of the plastics components to be fused is composed of thermoplastic; however, a coating of at least one part with a thermoplastic comprising at least one compound of the general formula (I) is required.

The plastics components employed for laser welding preferably comprise or consist of at least one polymer selected from polyolefins, polyolefin copolymers, polytetrafluoroethylenes, ethylene-tetrafluoroethylene copolymers, polyvinyl chlorides, polyvinylidene chlorides, polyvinyl alcohols, polyvinyl esters, polyvinyl alkanals, polyvinyl ketals, polyamides, polyimides, polycarbonates, polycarbonate blends, polyesters, polyester blends, poly(meth)acrylates, poly(meth)acrylate-styrene copolymer blends, poly(meth)acrylate-polyvinylidene difluoride blends, polyurethanes, polystyrenes, styrene copolymers, polyethers, polyether ketones and polysulfones and mixtures thereof.

Preference is given to matrix polymers from the group of the polyolefins, polyolefin copolymers, polyvinyl alkanals, polyamides, polycarbonates, polycarbonate-polyester blends, polycarbonate-styrene copolymer blends, polyesters, polyester blends, poly(meth)acrylates, poly(meth)acrylate-styrene copolymer blends, poly(meth)acrylate-polyvinylidene difluoride blends, styrene copolymers and polysulfones and mixtures thereof.

Particularly preferred polymers are transparent or at least translucent. Examples include: polypropylene, polyvinylbutyral, nylon-[6], nylon-[6,6], polycarbonate, polycarbonate-polyethylene terephthalate blends, polycarbonate-polybutylene terephthalate blends, polycarbonate-acrylonitrile/styrene/acrylonitrile copolymer blends, polycarbonate-acrylonitrile/butadiene/styrene copolymer blends, polymethyl methacrylate-acrylonitrile/butadiene/styrene copolymer blends (MABS), polyethylene terephthalate, polybutylene terephthalate, polymethyl methacrylate, impact-modified polymethyl methacrylate, polybutyl acrylate, polymethyl methacrylate-polyvinylidene difluoride blends, acrylonitrile/butadiene/styrene copolymers (ABS), styrene/acrylonitrile copolymers (SAN), polyphenylenesulfone and mixtures comprising 2 or more (e.g. 2, 3, 4, 5) of the afore-mentioned polymers.

Suitable polymer preparations for laser welding comprise
A) a thermoplastic matrix polymer suitable for forming the plastics parts,
B) at least one compound of the general formula (I) as defined before,
C) optionally at least one further additive.

Those polymer preparations for laser welding are likewise in accordance with the invention and are suitable for producing fusion-bonded plastics parts with the aid of laser radiation whose wavelength is outside the visible region.

Polymer preparations for laser welding may advantageously be produced by a conventional extrusion or kneading process. The components B), and, if present, C) may be mixed from the outset, in the weight ratio corresponding to the desired end concentration, with the matrix polymer A) (direct compounding), or a distinctly higher concentration of B) and, if present, C) may initially be selected and the concentrate formed (masterbatch) subsequently diluted with further matrix polymer A) in the course of the manufacture of the parts to be fused.

Suitable additives C) are UV stabilizers, antioxidants, processing plasticizers, etc.

In addition, the polymer preparations for laser welding may comprise at least one colorant for establishing a desired hue as additive, especially transparent organic pigments and in particular dyes, for example C.I. Pigment Yellow 138, 139, 147, 183, 185 192 and 196, C.I. Pigment Orange 70, C.I. Pigment Red 149,178 and 179, 181, 263, C.I. Pigment Violet 19 and 29, C.I. Pigment Blue 15, 15:1, 15:3 and 15:4, C.I. Pigment Green 7 and 36, C.I. Solvent Yellow 14, 21, 93, 130, 133, 145, 163, C.I. Solvent Red 52, 135, 195, 213, 214 and 225, C.I. Solvent Blue 35, 45, 67, 68, 97, 104, 122, 132, C.I. Solvent Violet 13, 46, 49, C.I. Solvent Green 3, 5 and 28, C.I. Solvent Orange 47, 60, 86, 114, and 163, C.I. Solvent Brown 35, 53, and also C.I. Disperse Yellow 54, 87, 201, C.I. Disperse Orange 30, C.I. Disperse Red 60 and C.I. Disperse Violet 57

A further possible additive group is that of additives which likewise modify the visual appearance, the mechanical properties or else the tactile properties, for example matting agents, such as titanium dioxide, chalk, barium sulfate, zinc sulfide, fillers, such as nanoparticulate silicon dioxide, aluminium hydroxide, clay and other sheet silicates, glass fibers and glass spheres.

Moreover, the compounds of formula (I) may also be used advantageously for laser marking and laser inscription. In this case, the laser light absorbed by the compound of formula (I) brings about heating of the plastic, which leads to it foaming or the conversion of a dye present in addition, and in this way gives rise to a marking or inscription.

Useful liquids which can be marked with the compounds of the formula (I) preferably include oils such as mineral oils, vegetable and animal fatty oils, and ethereal oils.

Examples of such oils are natural oils such as olive oil, soybean oil or sunflower oil, or natural or synthetic motor oils, hydraulic oils or transmission oils, for example motor vehicle oil or sewing machine oil, or brake fluids and mineral oils which, according to the invention, comprise gasoline, kerosene, diesel oil and also heating oil. Particular preference is given to mineral oils such as gasoline, kerosene, diesel oil or heating oil, in particular gasoline, diesel oil or heating oil. Particularly advantageously, the above-mentioned compounds of the formula (I) are used as markers for mineral oils in which labeling is simultaneously required, for example for tax reasons. In order to minimize the costs of labeling, but also in order to minimize possible interactions of the marked mineral oils with any other ingredients present, such as polyisobuteneamine (PIBA), efforts are made to minimize the amount of markers. A further reason to minimize the amount of markers may be to prevent their possible harmful influences, for example on the fuel intake and exhaust gas outlet region of internal combustion engines.

The compounds of the formula (I) to be used as markers are added to the liquids in such amounts that reliable detection is ensured. Typically, the (weight-based) total content of markers in the marked liquid is from about 0.1 to 5000 ppb, preferably from 1 to 2000 ppb and more preferably from 1 to 1000 ppb.

The compounds of the formula (I) may if appropriate also be used in a mixture with other markers/dyes.

To mark the liquids, the compounds are generally added in the form of solutions. Especially in the case of mineral oils, suitable solvents for providing these stock solutions are preferably aromatic hydrocarbons such as toluene, xylene or relatively high-boiling aromatics mixtures.

In the field of automated banknote processing, IR absorption plays an important role. Most of the actually circulating currency carries not only visibly coloured printings, but also specific features which are only detectable in the infrared part of the spectrum. Generally, these IR features are implemented for use by automatic currency processing equipment, in banking and vending applications (automatic teller machines, automatic vending machines, etc.), in order to recognize a determined currency bill and to verify its authenticity, in particular to discriminate it from replicas made by colour copiers.

Accordingly, the present invention also relates to a method of detecting the authenticity of a security document as defined above, or below, comprising the steps of:
a) measuring an absorbance spectrum of the security document in the NIR range of the electromagnetic spectrum; and
b) comparing the spectrum measured under a) and/or information derived therefrom with a corresponding spectrum and/or information of an authentic security element.

Some examples of three-dimensional (3D) printing may utilize a fusing agent (including an energy absorber) to pattern polymeric build material. The fusing agent is capable of absorbing radiation and converting the absorbed radiation to thermal energy, which in turn coalesces/fuses the polymeric build material that is in contact with the fusing agent.

Accordingly, the present invention is directed to fusing agents, comprising at least one compound of formula (I). The composition of the fusing agents is, for example, described in WO2020005200, WO2019245589, WO2019245518, WO2019245517, WO2019245535, WO2019245534, WO2019245516 and US2019382429.

In addition, the present invention is directed to a consumable material for use in an additive manufacturing system, the consumable material comprising:
at least one polymer comprising:
at least one compound of formula (I).

In addition, the present invention is directed to a consumable assembly for use in an extrusion-based additive manufacturing system, the consumable assembly comprising:
a container portion;
a consumable filament at least partially retained by the container portion, the consumable filament comprising:
at least one polymer, and
at least one compound of formula (I).

The consumable filament may have a core comprising the at least one polymer and a coating comprising at least one compound of formula (I) (WO2015130401).

The at least one polymer may be a meltable polymer which is selected from the group consisting of polyurethane, polyester, polyalkylene oxide, plasticized PVC, polyamide, protein, PEEK, PEAK, polypropylene, polyethylene, thermoplastic elastomer, POM, polyacrylate, polycarbonate, polymethylmethacrylate, polystyrene or a combination of at least two of these.

A process for producing an article by means of an additive manufacturing method from the consumable material comprises at least temporarily exposing the consumable material to infrared radiation in the wavelength range between 600 nm and 1700 nm.

The present invention is also directed to an article obtainable by the process.

Various features and aspects of the present invention are illustrated further in the examples that follow. While these examples are presented to show one skilled in the art how to operate within the scope of this invention, they are not to serve as a limitation on the scope of the invention where such scope is only defined in the claims. Unless otherwise indicated in the following examples and elsewhere in the specification and claims, all parts and percentages are by weight, temperatures are in degrees centigrade and pressures are at or near atmospheric.

### EXAMPLES

### Example 1

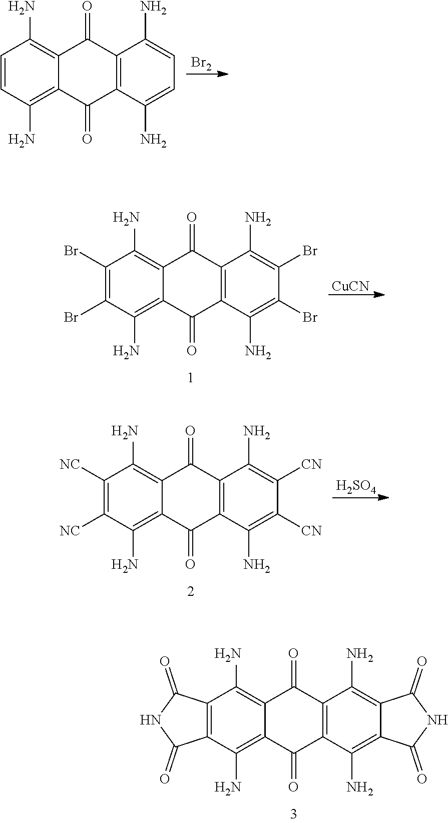

### a) 2,3,6,7-tetrabromo-1,4,5,9-tetramino-9,10-anthraquinone (1)

In a 500 mL round-bottomed flask, bromine (9 mL, 176 mmol) was added dropwise over 5 minutes to a solution of 1,4,5,9-tetraamino-9,10-anthraquinone (3 g, 11 mmol) in acetic acid (250 mL) at room temperature. After heating at 110° C. for 6 h, the solution was allowed to cool down and poured into 500 mL water. NaHSO₃ (1.76 g) was added while stirring to quench the excessive bromine. The as-obtained solid was then filtered and washed with 200 mL K₂CO₃ aqueous solution (1 mol/L) to neutralize the pH. After oven drying at 100° C. for 1 h, compound (1) was collected as a purple solid (5.8 g, 90%). HRMS (APPI+) m/z: calcd for C₁₄H₈Br₄N₄O₂ [M⁺]: 579.7381; Found: 579.7350.

### b) 2,3,6,7-tetracyano-1,4,5,9-tetramino-9,10-anthraquinone (2)

In a 500 mL round-bottomed flask, compound 1 (600 mg, 1 mmol) and CuCN (2 g, 22 mmol) were mixed in NMP (250 mL) with continuous N₂ bubbling for 15 min. The solution was then heated to 150° C. for 24 h. After cooling down to room temperature, 500 mL water was added to precipitate out the product. The as-obtained solid was then filtered and washed with 200 mL water and 100 mL methanol, successively. After oven drying at 100° C. for 1 h, compound (2) was collected as a green solid (4 g, 90%). HRMS (MALDI+) m/z: calcd for C₁₈H₈N₈O₂ [M⁺]: 368.0770; Found: 368.0771.

### c) 1,4,5,9-tetramino-2,3,6,7-9,10-anthraquinone diimide (3)

In a 250 mL round-bottomed flask, compound 2 (2 g) was slowly added into concentrated sulfuric acid (100 mL) with vigorous stirring at 80° C. The solution was then heated to 150° C. and stirred for 1.5 h. After cooling down to room temperature, 500 mL iced water was added to precipitate out the crude product. The as-obtained solid was then filtered and washed with 200 mL water and methanol, successively. The as-obtained solid was dissolved in NMP (200 mL) at 150° C. and stirred for 15 minutes. The solution was filtered while still hot. 1 L water was added into the filtrate to precipitate the product. After filtration and oven drying at 100° C. for 1 h, compound (3) was collected as a blue solid (1 g, 50%). ¹H NMR (500 MHz, DMSO-d6, 393 K, ppm): δ 10.48 (s, 2H, NH), 7.44 (br, 8H, NH2). HRMS (MALDI+) m/z: calcd for C₁₈H₁₀N₆O₆[M+]: 406.0656; Found: 406.0662. Elem. Anal. Calcd for C₁₈H₁₀N₆O₆: C, 53.21; H, 2.48; N, 20.68. Found: C, 52.21; H, 2.56; N, 20.70. IR (KBr, cm⁻¹): 3420 s, 1727 s, 1649 s. UV/Vis (THF): λₘₐₓ=802 nm (ε=18390 M⁻¹cm⁻¹). Mp>300° C.

### Example 2

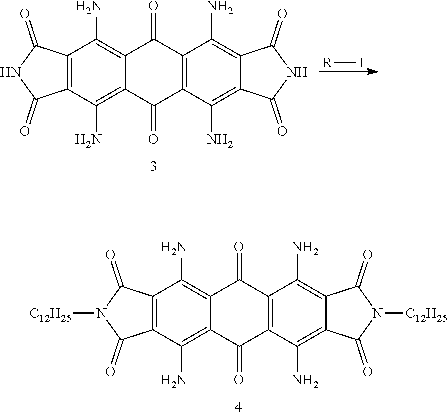

### N,N′-Bisdodecyl-1,4,5,9-tetramino-2,3,6,7-9,10-anthraquinone diimide (4)

In a 100 mL round-bottomed flask, compound 3 (406 mg, 1 mmol) and NaH (120 mg, 5 mmol) were mixed in DMSO (50 mL) with continuous N₂ bubbling for 15 min. To the mixture, 1-iodododecane (1200 mg, 4 mmol) was added dropwise over 5 min at room temperature. After stirring at room temperature for 12 h, 100 mL water was poured into the solution. The resulting precipitate was then filtered and washed with 100 mL water. After oven drying at 100° C. for 1 h, the title compound was collected as a blue solid (660 mg, 90%). ¹H NMR (400 MHz, THF-d8, 293 K, ppm): δ 10.72 (s, 8H, NH2), 1.24 (5, 36H, J=6.0 Hz, CH2), 0.80 (m, 6H, CH3). HRMS (MALDI+) m/z: calcd for C42H58N6O6 [M+]: 742.9465; Found: 742.9566.

Elem. Anal. Calcd for C42H58N6O6: C, 67.90; H, 7.87; N, 11.31. Found: C, 68.20; H, 7.91; N, 11.60. IR (KBr, cm-1): 3443 s, 2921 s, 2851 s, 1042 s. UV/Vis (THF): λₘₐₓ=794 nm (ε=28600 M⁻¹cm⁻¹). Mp>300° C.

Compound (4) shows good solubility in THF. A solution of compound (4) is colorless due to weak absorption in visible region.

1.-15. (canceled)

16. A compound of the formula 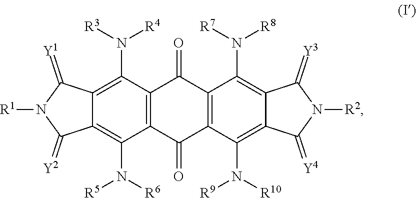
wherein
Y¹, Y², Y³ and Y⁴ are O, or one of Y¹ and Y² is O and the other is NR¹³ and one of Y³ and Y⁴ is O and the other is NR¹, wherein R¹³ has the meaning of R¹;
especially 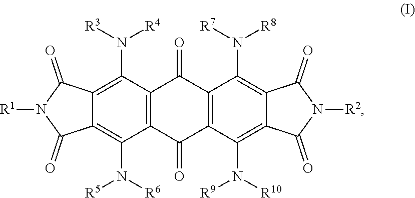
wherein
R¹ and R² are independently of each other selected from hydrogen, C₁-C₂₄-alkyl, C₁-C₂₄-haloalkyl, C₃-C₂₄-cycloalkyl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with one or more substituents R¹¹, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from O, S and NR¹²;
R³, R⁴, R⁵, R⁶, R⁷, R⁸, R⁹ and R¹⁰ are independently of each other selected from hydrogen, C₁-C₂₄-alkyl, C₁-C₂₄-haloalkyl, C₃-C₂₄-cycloalkyl, heterocycloalkyl, hetaryl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with one or more substituents R^{11′}, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from O, S and NR^{12′};
each R¹¹ is selected from C₁-C₂₄-alkyl, C₁-C₂₄-fluoroalkyl, C₁-C₂₄-alkoxy, fluorine, chlorine or bromine;
each R^{11′} is selected from C₁-C₂₄-alkyl, C₁-C₂₄-fluoroalkyl, C₁-C₂₄-alkoxy, fluorine, chlorine or bromine; and
R¹² and R^{12′} are independently of each other hydrogen, C₁-C₂₀-alkyl, C₃-C₂₄-cycloalkyl, heterocycloalkyl, hetaryl or C₆-C₁₀-aryl.

17. The compound according to claim 16, which is a compound of formula 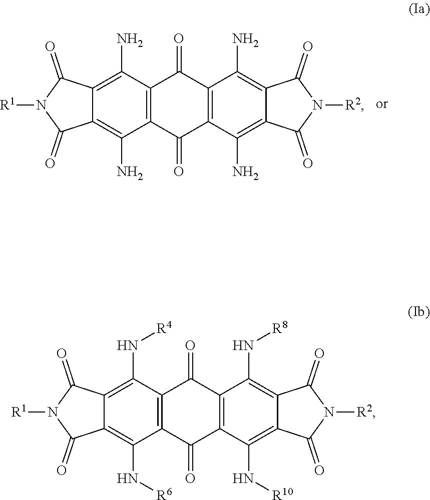
wherein
R⁴, R⁶, R⁸, R¹⁰, R¹ and R² are defined in claim 16.

18. The compound according to claim 16, wherein R¹ and R² are independently of each other selected from hydrogen, linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹¹, wherein R¹¹ is defined in claim 16.

19. The compound according to claim 16, wherein R³, R⁴, R⁵, R⁶, R⁷, R⁸, R⁹ and R¹⁰ are independently of each other selected from hydrogen, C₁-C₂₄-alkyl, C₃-C₂₄-cycloalkyl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with 1, 2 or 3 identical or different substituents R^{11′}, wherein R^{11′} is defined in claim 16.

20. The compound according to claim 16, which is a compound of formula 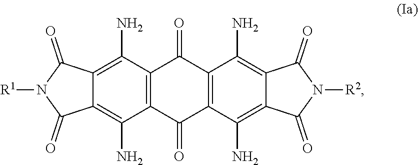
wherein
R¹ and R² are identical and are selected from hydrogen, linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹¹, wherein R¹¹ is C₁-C₁₂-alkyl, or C₁-C₁₂-alkoxy.

21. The compound according to claim 16, which is a compound of formula 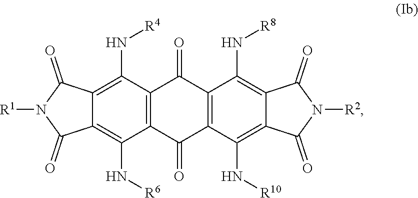
wherein
R¹ and R² are identical and are selected from hydrogen, linear C₁-C₂₄-alkyl, branched C₃-C₂₄-alkyl, C₅-C₈-cycloalkyl, phenyl and phenyl-C₁-C₁₀-alkylene, where the rings of cycloalkyl, phenyl and phenyl-alkylene in the three last-mentioned radicals are unsubstituted or substituted by 1, 2 or 3 identical or different substituents R¹¹, wherein R¹¹ is C₁-C₆-alkyl, or C₁-C₆-alkoxy; and
R⁴, R⁶, R⁸ and R¹⁰ are identical and are selected from C₁-C₂₄-alkyl, C₃-C₂₄-cycloalkyl, phenyl and phenyl-C₁-C₄-alkylene, where the rings of cycloalkyl, phenyl, and phenyl-alkylene are unsubstituted or substituted with 1, 2 or 3 identical or different substituents R^{11′} wherein R^{11′} is C₁-C₆-alkyl, or C₁-C₆-alkoxy.

22. The compound according to claim 16, which is
a compound of formula

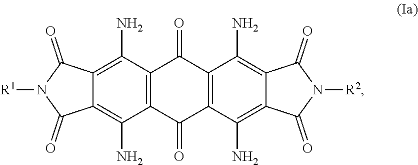

| Cpd. | R¹ = R² |
|---|---|
| 3 | H |
| 4 | —C₁₂H₂₅ |
| 4a | (C₁₀H₂₁)₂CH— |
| 4b | 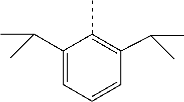 |
| 4c | 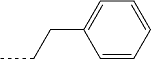 |
| 4d | 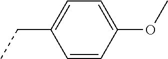 |
| 4e | —CH₂CH(C₂H₅)CH₂CH₂CH₂CH₃ |
| 4f | 3-propyl heptyl |
| 4g | (C₈H₁₇)₂CH— |
| 4h | (C₆H₁₃)₂CH— |

or a compound of formula

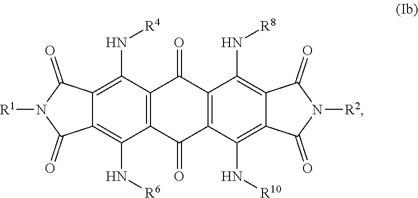

| Cpd. | R⁴ = R⁶ = R⁸ = R¹⁰ | R¹ = R² |
|---|---|---|
| 5a1 | n-butyl | —C₁₂H₂₅ |
| 5a2 | ″ | (C₁₀H₂₁)₂CH— |
| 5a3 | ″ | 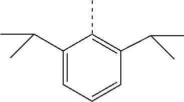 |
| 5a4 | ″ | 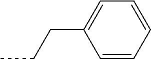 |
| 5a5 | ″ | 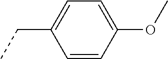 |
| 5a6 | ″ | —CH₂CH(C₂H₅)CH₂CH₂CH₂CH₃ |
| 5a7 | ″ | 3-propyl heptyl |
| 5a8 | ″ | (C₈H₁₇)₂CH— |
| 5a9 | ″ | (C₆H₁₃)₂CH— |
| 5a10 | ″ | H |
| 5b1 | 4-butylphenyl | —C₁₂H₂₅ |
| 5b2 | ″ | (C₁₀H₂₁)₂CH— |
| 5b3 | ″ | 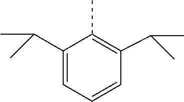 |
| 5b4 | ″ | 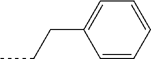 |
| 5b5 | ″ | 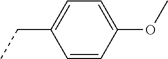 |
| 5b6 | ″ | —CH₂CH(C₂H₅)CH₂CH₂CH₂CH₃ |
| 5b7 | ″ | 3-propyl heptyl |
| 5b8 | ″ | (C₈H₁₇)₂CH— |
| 5b9 | ″ | (C₆H₁₃)₂CH— |
| 5b10 | ″ | H |
| 5cl | 2,4,6-trimethylphenyl | —C₁₂H₂₅ |
| 5cl | ″ | —C₁₂H₂₅ |
| 5c2 | ″ | (C₁₀H₂₁)₂CH— |
| 5c3 | ″ | 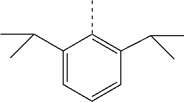 |
| 5c4 | ″ | 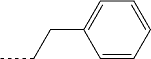 |
| 5c5 | ″ | 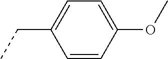 |
| 5c6 | ″ | —CH₂CH(C₂H₅)CH₂CH₂CH₂CH₃ |
| 5c7 | ″ | 3-propyl heptyl |
| 5c8 | ″ | (C₈H₁₇)₂CH— |
| 5c9 | ″ | (C₆H₁₃)₂CH— |
| 5c10 | ″ | H |
| 5cl | ″ | —C₁₂H₂₅ |
| 5d1 | cyclohexyl | —C₁₂H₂₅ |
| 5d2 | ″ | (C₁₀H₂₁)₂CH— |
| 5d3 | ″ | 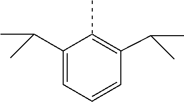 |
| 5d4 | ″ | 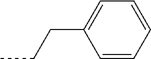 |
| 5d5 | ″ | 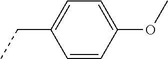 |
| 5d6 | ″ | —CH₂CH(C₂H₅)CH₂CH₂CH₂CH₃ |
| 5d7 | ″ | 3-propyl heptyl |
| 5d8 | ″ | (C₈H₁₇)₂CH— |
| 5d9 | ″ | (C₆H₁₃)₂CH— |
| 5d10 | ″ | H |
| 5e1 | n-pentyl | —C₁₂H₂₅ |
| 5e2 | ″ | (C₁₀H₂₁)₂CH— |
| 5e3 | ″ | 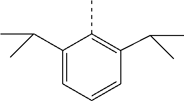 |
| 5e4 | ″ | 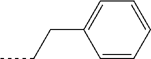 |
| 5e5 | ″ | 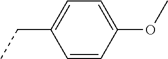 |
| 5e6 | ″ | —CH₂CH(C₂H₅)CH₂CH₂CH₂CH₃ |
| 5e7 | ″ | 3-propyl heptyl |
| 5e8 | ″ | (C₈H₁₇)₂CH— |
| 5e9 | ″ | (C₆H₁₃)₂CH— |
| 5e10 | ″ | H |
| 5f1 | methyl | —C₁₂H₂₅ |
| 5f2 | ″ | (C₁₀H₂₁)₂CH— |
| 5f3 | ″ | 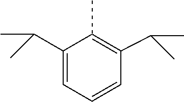 |
| 5f4 | ″ | 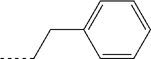 |
| 5f5 | ″ | 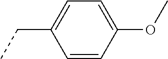 |
| 5f6 | ″ | —CH₂CH(C₂H₅)CH₂CH₂CH₂CH₃ |
| 5f7 | ″ | 3-propyl heptyl |
| 5f8 | ″ | (C₈H₁₇)₂CH— |
| 5f9 | ″ | (C₆H₁₃)₂CH— |
| 5f10 | ″ | H |
| 5g1 | 2-ethyl hexyl | —C₁₂H₂₅ |
| 5g2 | ″ | (C₁₀H₂₁)₂CH— |
| 5g3 | ″ | 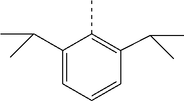 |
| 5g4 | ″ | 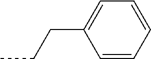 |
| 5g5 | ″ | 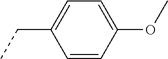 |
| 5g6 | ″ | —CH₂CH(C₂H₅)CH₂CH₂CH₂CH₃ |
| 5g7 | ″ | 3-propyl heptyl |
| 5g8 | ″ | (C₈H₁₇)₂CH— |
| 5g9 | ″ | (C₆H₁₃)₂CH— |
| 5g10 | ″ | H |

23. An TR absorber for optical filter applications, for plasma display panels, for laser marking of paper or plastics, for laser welding of plastics, for 3D printing, for the curing of surface-coatings using TR radiators, for the drying and curing of print, for the fixing of toners on paper or plastics, for heat shielding applications, for invisible or JR readable bar codes, as JR absorber in security printing or for brand protection, or as marker for liquids, especially oils, comprising the compound of claim 16.

24. A printing ink formulation, comprising at least one compound according to claim 16.

25. A printing ink formulation, comprising
a) at least one compound according claim 16,
b) a polymeric binder,
c) a solvent,
d) optionally at least one colorant, and
e) optionally at least one further additive.

26. A process for the manufacture of a security document comprising the steps printing on a substrate a printing ink formulation according to claim 24.

27. A security document, comprising a substrate and at least one compound according to claim 16.

28. A method of detecting the authenticity of the security document as defined in claim 27, comprising the steps of:
a) measuring an absorbance, reflectance or transmittance spectrum of the security document in the NIR range of the electromagnetic spectrum; and
b) comparing the spectrum measured under a) or information derived therefrom with a corresponding spectrum or information of an authentic security element.

29. A process for the preparation of a compound of formula 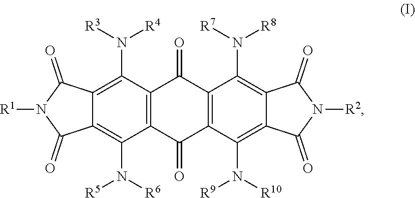
wherein
R¹, R², R³, R⁴, R⁵, R⁶, R⁷, R⁸, R⁹ and R¹⁰ are defined in claim 16, comprising the following steps:
i) heating a compound of formula 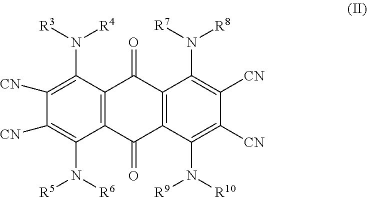
in H₂SO₄, to obtain a compound of formula (I), wherein R¹ and R² are hydrogen, and
ii) optionally reacting the compound obtained in step i) with a compound R¹—X¹ in the presence of a base to obtain a compound of formula (I), wherein X¹ is Cl, Br, or I and R¹ and R² are different from hydrogen.

30. The process according to claim 29, wherein the compound of formula 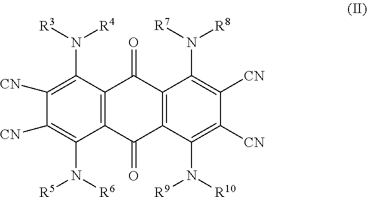
is obtained by reacting a compound of formula 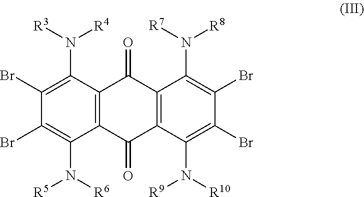
with CuCN in a solvent at elevated temperature, wherein R³, R⁴, R⁵, R⁶, R⁷, R⁸, R⁹ and R¹⁰ are independently of each other selected from hydrogen, C₁-C₂₄-alkyl, C₁-C₂₄-haloalkyl, C₃-C₂₄-cycloalkyl, heterocycloalkyl, hetaryl, C₆-C₁₀-aryl and C₆-C₁₀-aryl-C₁-C₁₀-alkylene, where the rings of cycloalkyl, aryl, and aryl-alkylene in the three last-mentioned radicals are unsubstituted or substituted with one or more substituents R^{11′}, and where C₁-C₂₄-alkyl, C₁-C₂₄-haloalkyl and the alkylene moiety of C₆-C₁₀-aryl-C₁-C₁₀-alkylene may be interrupted by one or more heteroatoms or heteroatomic groups selected from 0, S and NR^{12′};
each R^{11′} is selected from C₁-C₂₄-alkyl, C₁-C₂₄-fluoroalkyl, C₁-C₂₄-alkoxy, fluorine, chlorine or bromine; and
R^{12′} is hydrogen, C₁-C₂₀-alkyl, C₃-C₂₄-cycloalkyl, heterocycloalkyl, hetaryl or C₆-C₁₀-aryl.
